(19) United States
(12) Patent Application Publication
(10) Pub. No.: US 2008/0243194 A1
(43) Pub. Date: Oct. 2, 2008

(54) ARTICULATING INSTRUMENTATION FOR DYNAMIC SPINAL STABILIZATION

(75) Inventors: Jeffrey C. Lotz, San Mateo, CA (US); David S. Bradford, Sausalito, CA (US)

Correspondence Address: JOHN P. O'BANION;O'BANION & RITCHEY LLP, 400 CAPITOL MALL SUITE 1550, SACRAMENTO, CA, 95814, US

(73) Assignee: THE REGENTS OF THE UNIVERSITY OF CALIFORNIA, Oakland, CA (US)

(21) Appl. No.: 12/055,531

(22) Filed: Mar. 26, 2008

Related U.S. Application Data
(63) Continuation-in-part of application No. PCT/US2006/037479, filed on Sep. 26, 2006.
(60) Provisional application No. 60/720,830, filed on Sep. 26, 2005.
(60) Provisional application No. 60/908,652, filed on Mar. 28, 2007.

(51) Int. Cl. A61F 5/00; A61B 17/70; A61B 17/04

(52) U.S. Cl. 606/86 A; 606/246; 606/301

(57) ABSTRACT

Dynamic stabilization apparatus and methods in the context of a spinal implant configured to constrain intervertebral movement, where the constraint is meant to eliminate unwanted, non-physiologic motions. The system provides dynamic stability to motion in a compromised spinal joint by allowing motion along a centrode of the instant axis of rotation (IAR) that substantially approximates the normal centrode for the respective spinal joint. The system and method is adapted to provide stabilized motion in a spinal joint such that the IAR shifts cephalad during typical flexion ranges beyond a normal resting range of motion, and shifts posteriorly during typical extension ranges beyond the normal resting range of motion.

### CROSS-REFERENCE TO RELATED APPLICATIONS

This application claims priority from, and is a continuation-in-part of, co-pending PCT international application serial number PCT/US2006/037479, filed on Sep. 26, 2006, incorporated herein by reference in its entirety, which claims priority from U.S. provisional application Ser. No. 60/720,830, filed on Sep. 26, 2005, incorporated herein by reference in its entirety, and this application claims priority from U.S. provisional application Ser. No. 60/908,652, filed on Mar. 28, 2007, incorporated herein by reference in its entirety,

This application is also related to PCT International Publication No. WO 2007/038510, published on Apr. 5, 2007, incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND OF THE INVENTION

1. Field of the Invention

This invention pertains generally to spine stabilization instruments, and more particularly to dynamic spine stabilization instruments.

2. Description of Related Art

Degenerative disc disease is an important public health problem with multiple dimensions: personal, social, and professional. It is also well recognized that facet arthritis is associated with disc degeneration, and this is typically attributed to loss of disc height and consequently increased posterior column loads. However, in addition to disc height loss, intervertebral kinematics becomes progressively erratic with increasing disc degeneration, being characterized by significant variability in the instantaneous axis of rotation (IAR) position (centrode). Since spinal movement is constrained by both the disc and facet joints, disc material property deterioration with degeneration also influences facet forces. Unfortunately, the influence of IAR position fluctuations on facet loads, and consequently arthritis risk, has not been previously investigated or reported.

There is a growing acknowledgement that back pain patients who are surgical candidates will benefit over the long term from less invasive procedures that facilitate dynamic stabilization, rather than fusion. The under-riding philosophy is that morbidity from the surgical technique or accelerated degeneration at adjacent segments, ultimately limit the success of current fusion procedures.

Dynamic stabilization can take on many forms, from those providing assistance using mechanical devices (e.g. partial disc replacement, posterior dynamic stabilization), to those relying on biologic processes (tissue regeneration/repair).

During spinal movement, intervertebral motion is relative to a variable instant axis of rotation (IAR). Several current forms of dynamic stabilization attempt to facilitate motion about a single or variable axis of rotation. For example, total disc replacements serve to substitute the intervertebral disc with an articulating implant that guides motion. Or, posterior instrumentation previously designed for rigid fusion has been modified to include flexible members that allow some degree of intervertebral movement. Unfortunately, these devices don't necessarily replicate the natural IAR and as a consequence may lead to fact overload, facet arthritis, and back pain.

Posterior dynamic stabilization has the advantage of leaving the disc space intact and being facilitated by a less invasive surgical procedure. Current posterior dynamic stabilization technologies are incremental improvements of traditional rod and screw fusion systems that incorporate either flexible rods or articulating rod/screw attachments. These systems however, do not support the natural IAR.

Recently, advances in surgical technique and instrumentation have generated interest in disc arthroplasty as a novel technique for treating degenerative disc disease. Different intervertebral implant designs have been used for restoring disc height and painless motion. As with disc degeneration, disc replacement alters the disc/facet synergy in yet, unknown ways. Consequently, the influence of many implant design choices, such as the degree of constraint, bearing surface shape, and size, may alter facet forces and the patients risk for developing facet arthritis.

Because of its caudal location and its crucial role in spine sagittal balance, the L5/S1 joint is one of the most commonly degenerated levels and the most common site of disc replacement for degenerative disc disease. Due to the sagittal obliquity of the sacral endplate, anterior intervertebral shear is significant at this level. Consequently, the facet joints are critical for preventing spondylolisthesis and constraining inter-segmental motion.

Certain aspects of L5/S1 kinematics in vivo and in vitro under different loading conditions have been previously observed. Certain forces transmitted through the facet joints in various intervertebral positions under pure axial compression have also been previously reported. These previous in vivo studies were limited, however, by not measuring facet forces. Moreover, the previous in vitro studies were limited by presenting only simplified and non-physiologic loading conditions by omitting to account for the fact that, in addition to compression, the L5/S1 level supports significant anterior shear. Given that the disc is viscoelastic and spinal kinematics can vary with the magnitude and nature of superimposed loading, previous studies thus missed clinically-relevant interactions between the kinematics and facet forces.

As a consequence, previous attempts at providing artificial dynamic stabilization tools and methods have not accurately addressed the desired spatial ranges of spinal motion, resulting in tools and methods that present certain inadequacies and shortcomings with direct medical consequences.

Consequently, a need still exists for a system and method for restoring compromised spinal disc joints to a more natural instant axis of rotation (IAR).

### BRIEF SUMMARY OF THE INVENTION

An aspect of the invention is a method of stabilizing adjacent vertebrae. The method includes the steps of installing a first anchor in a first vertebra and a second anchor in a second vertebra adjacent to said first vertebra, and coupling an articulating linkage to said first and second anchors. The articulating linkage constrains one or more components of motion between the first and second vertebrae while allowing the first vertebra to move along the path of the IAR of the first vertebra. In a preferred embodiment, the linkage is configured to constrain non-physiologic motion between the first and second vertebrae.

Generally, the IAR of the first vertebra comprises an axis that the first vertebra rotates about and travels along as it moves from one position to another.

Coupling an articulating linkage may be achieved by attaching a first member to the first anchor and a second member to the second anchor, and establishing one or more hinges about one or more respective pivot points, wherein the one or more hinges link the first member to the second member, and wherein the one or more pivot points correlate to the IAR of the first vertebra.

In one embodiment, the first member is coupled to the second member via a first articulating link having a first pivot point on the first member and a second pivot point on the second member, and a second articulating link having a third pivot pint on the first member and a fourth pivot point on the second member.

In a preferred embodiment, the first member and first anchor are rigidly fixed to each other such that they move in unison along with the first vertebra. Correspondingly, the second member and second anchor are rigidly fixed to each other such that they move in unison along with the second vertebra.

The first or second anchor may comprise any one of known fastening means available in the art, such as a pedicle screw installed in a pedicle of the vertebra.

In one embodiment, the linkage is installed in a posterior region of the vertebrae.

In another embodiment, the first vertebra comprises the L5 vertebra, and the second vertebra comprises the S1 vertebra. Preferably, the articulating linkage allows the L5 vertebra to rotate and translate with respect to the S1 vertebra. In addition, the rotation and translation of the L5 vertebra follows that path of the IAR of the L5 vertebra. More particularly, the L5 IAR intersection with the midsagittal plane moves cephalid relative to the S1 endplate during flexion, and posterior during extension. In some embodiments, the articulating linkage is configured to allow the L5 vertebra to rotate substantially forward during flexion, and substantially backward during extension.

Another aspect of the invention is an apparatus for stabilizing adjacent vertebrae. The apparatus includes a first anchor configured to be installed in a first vertebra, a second anchor configured to be installed in a second vertebra adjacent to said first vertebra, and an articulating linkage coupling said first and second anchors. The articulating linkage is configured to constrain one or more components of motion between the first and second vertebrae while allowing the first vertebra to move along the path of the IAR of the first vertebra.

In one embodiment, the articulating linkage comprises first and second members configured to be attached to the first and second anchors respectively, and one or more hinges centered about one or more respective pivot points, wherein the one or more hinges link the first member to the second member, and the one or more pivot points correlate to the IAR of the first vertebra.

Another aspect is an apparatus for dynamically stabilizing adjacent vertebrae. The apparatus comprises a superior anchor configured to be installed in a superior vertebra, an inferior anchor configured to be installed in an inferior vertebra adjacent to the superior vertebra, and means for rotatably linking the superior anchor with the inferior anchor such that one or more components of motion between the superior and inferior vertebrae are constrained while allowing the superior vertebra to move along the path of the IAR of the superior vertebra.

In one embodiment, the linking means is configured to constrain non-physiologic motion between the first and second vertebrae. Preferably, the linking means articulates about one or more pivot points that correlate to the IAR of the first vertebra.

In another embodiment, the superior anchor comprises a superior pedicle screw configured to be installed in a pedicle of the superior vertebra. Correspondingly, the inferior anchor comprises an inferior pedicle screw configured to be installed in a pedicle of the inferior vertebra. For example, the superior vertebra comprises the L5 vertebra and the inferior vertebra comprises the S1 vertebra. The linking means is configured to allow the L5 vertebra to rotate and translate with respect to the S1 vertebra. Ideally, the rotation and translation of the L5 vertebra follows that path of the IAR of the L5 vertebra. The linking means may be configured to allow the L5 vertebra to rotate substantially forward during flexion, and substantially backward during extension.

Another aspect is an apparatus for stabilizing first and second adjacent vertebrae, comprising a dynamic stabilization assembly configured to be implanted in relation to the first and second vertebrae, wherein the first and adjacent vertebra comprise a vertebral joint having at least one IAR associated with the first and second vertebrae. The dynamic stabilization assembly is configured to allow at least a portion of the vertebral joint to rotate substantially about a first IAR corresponding to a first range of motion associated with the vertebral joint.

In one embodiment of the current aspect, the dynamic stabilization assembly is further configured to allow at least a portion of the vertebral joint to rotate substantially about a second IAR corresponding to a second range of motion associated with the vertebral joint.

Generally, the first IAR and second IAR have different locations with respect to a disc plane associated with the vertebral joint. In one embodiment, the position of the first IAR with respect to the second IAR shifts substantially laterally across the disc plane during the first range of motion. In another embodiment, the position of the first IAR with respect to the second IAR shifts substantially vertically along a spinal axis of the first and second vertebrae during the second range of motion.

In yet another embodiment, the dynamic stabilization assembly comprises a plurality of members coupled to the first and second vertebrae, wherein the plurality of members are configured to constrain motion of the vertebral joint while allowing at least a portion of the vertebral joint to move in accordance with the IAR. For example, the plurality of members may comprise a four-bar linkage, or other type of dynamic stabilization constraint that allows motion in accordance with the IAR. For example, the linkage may comprise a plurality of pivot points associated with the IAR.

Another aspect of the invention is a method for stabilizing first and second adjacent vertebrae. The method includes the steps of implanting a dynamic stabilization assembly in relation to the first and second vertebrae, wherein the first and adjacent vertebra comprise a vertebral joint having at least one IAR associated with the first and second vertebrae. The method further includes restraining motion of the vertebral joint while allowing at least a portion of the vertebral joint to rotate substantially about a first IAR corresponding to a first range of motion associated with the vertebral joint.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIGS. 1A and 1B show a graphical demonstration of the orientation of the instant axis of rotation for five sectors of flexion/extension (FIG. 1A) and lateral bending (FIG. 1B).

FIGS. 2A and 2B show the intersection of the axes illustrated in FIGS. 1A and 1B with the sagittal plane (FIG. 2A) and frontal plane (FIG. 2B) are superimposed on the L5/S1 spinal segment.

FIG. 3 shows an example of link points for a four-bar linkage that generates normal flexion/extension motion for the L5/S1 interspace superimposed over an L5/S1 spinal joint.

FIG. 4 shows a schematic representation of posterior dynamic stabilization linkage instrumentation in lateral view superimposed over FIG. 3.

FIGS. 5A-C show three positions of the schematic representation of the posterior dynamic stabilization assembly shown in FIG. 4, demonstrating its ability to guide L5 through a physiologic flexion/extension movement.

FIG. 6 illustrates the L5/S1 joint and respective coordinate system.

FIG. 7 shows a schematic diagram of L5/S1, and shows 40° sacral slope and 850 N load in standing position

FIG. 8 shows testing device with wedge to simulate constrained L5 posture in flexion, extension, and bending for investigating L5/S1 kinematics.

FIGS. 9A and 9B show schematic lateral view of L5/S1 facets, with facets open into flexion when the IAR is above the facet level (FIG. 9A), and facets close into flexion when the IAR is below the facet level (FIG. 9B).

FIG. 10 shows a graph of IAR distance to S1 endplate (zᵢ: mm) plotted against facet force variation (N) for each 3° rotation into flexion.

FIGS. 11a-c show a schematic side view of a further dynamic spinal stabilization embodiment, during different modes of use corresponding with different ranges of motion.

FIGS. 12a-c show a schematic side view of another dynamic spinal stabilization embodiment, during different modes of use corresponding with different ranges of motion.

FIG. 13 shows a schematic side view of another dynamic stabilization embodiment.

FIGS. 14a-c show a schematic side view of another dynamic spinal stabilization assembly during different respective modes of use corresponding with different ranges of motion.

FIG. 15a shows a schematic side view of another dynamic spinal stabilization embodiment.

FIG. 15b shows a schematic cross-sectioned view of a portion of the embodiment shown in FIG. 15a.

### DETAILED DESCRIPTION OF THE INVENTION

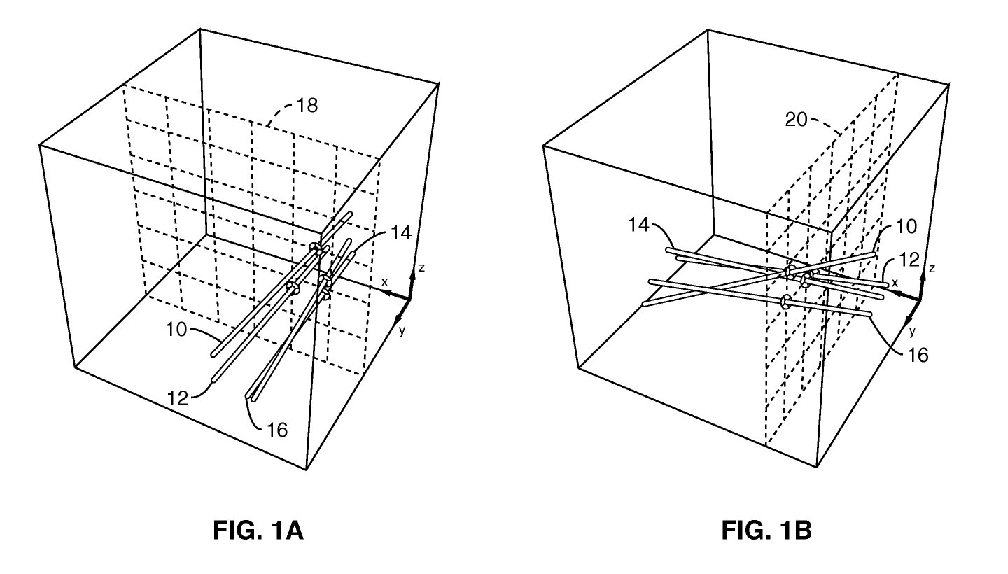

Referring more specifically to the drawings, for illustrative purposes the present invention is embodied in the apparatus generally shown in FIG. 1 through FIG. 15b. It will be appreciated that the apparatus may vary as to configuration and as to details of the parts, and that the method may vary as to the specific steps and sequence, without departing from the basic concepts as disclosed herein.

As is made more clear by way of illustration according to the various detailed embodiments herein described, these inadequacies and shortcomings are significantly overcome according to various aspects of the present invention.

It is to be appreciated that the present embodiments of the invention relate to providing improved dynamic stabilization in compromised spinal disc joints. In particular, the present embodiments provide informed solutions as to new experimental methods and observations that have shed new light on the desired performance that artificial dynamic stabilization demonstrate to more closely approximate normal spinal motion. In particular, the present embodiments more precisely approximate the IAR path (centrode) for normal spinal motion, which has been newly observed in experiments described herein to migrate in three dimensions. Further aspects of these particularly enlightened parameters for dynamic spinal motion are described in additional detail as follows.

According to one aspect, a dynamic stabilization system for use in providing dynamic stability to motion in a compromised spinal joint is provided. This system is adapted to provide a centrode of the instant axis of rotation (IAR) that substantially approximates the normal centrode for the respective spinal joint.

According to one mode of this aspect, the system is adapted to provide a centrode for the IAR for the respective joint that remains within a range of error of about 25 percent versus the normal centrode. In another mode, the range of error is within about 10 percent of the normal centrode.

According to another mode of this aspect, the system is adapted to provide a change in IAR that, during one range of motion of the spinal joint is principally lateral, or horizontal across the disc plane, and in another range of motion of the spinal joint is principally vertical along the spinal axis between vertebral bodies adjoining the disc of the joint.

Other aspects, modes, embodiments, features, and variations will become apparent to one of ordinary skill based upon a detailed review of this disclosure in its entirety and in the context of other information herein incorporated by reference or otherwise available.

Turning now to the figures and recitation to more detailed exemplary embodiments of the various broad aspects of the invention, FIG. 1 shows the natural centrode location of IAR for five postures (lateral view shown on the left, and AP view shown on the right) according to certain experimental test parameters described in further detail below.

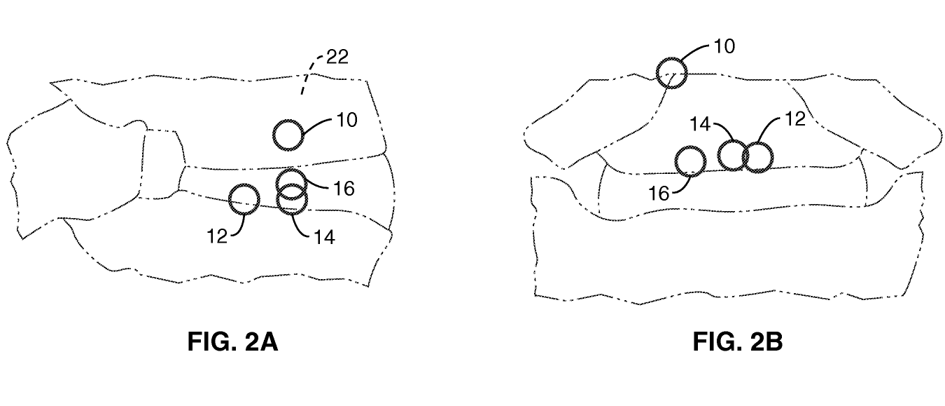

FIGS. 1A and 1B illustrate a 3-dimensional view of the orientation of the instant axis of rotation (IAR) for five sectors of flexion/extension (FIG. 1A) and lateral bending (FIG. 1B) in a L5/S1 vertebral joint. Axes 10 are the IAR for 30 to 6° flexion, axes 12 are the IAR for 3° to 6° extension, axes 14 are the IAR for 3° to neutral extension, and axes 16 are the IAR 3° to neutral flexion. The intersection of these axes with the sagittal plane 18 and frontal plane 20 are superimposed on the L5/S1 spinal segment (showing L5 (lumbar) vertebra 22, and S1 (sacrum) vertebra 24) in FIGS. 2A and 2B respectively. As can be seen in FIG. 2A, the natural centroid was found to be posteriorly located in extension (centroid 12), and more anteriorly and superiorly located in flexion (centroid 10). Accordingly, these centroid are provided by the dynamic stabilization devices and methods of the present invention

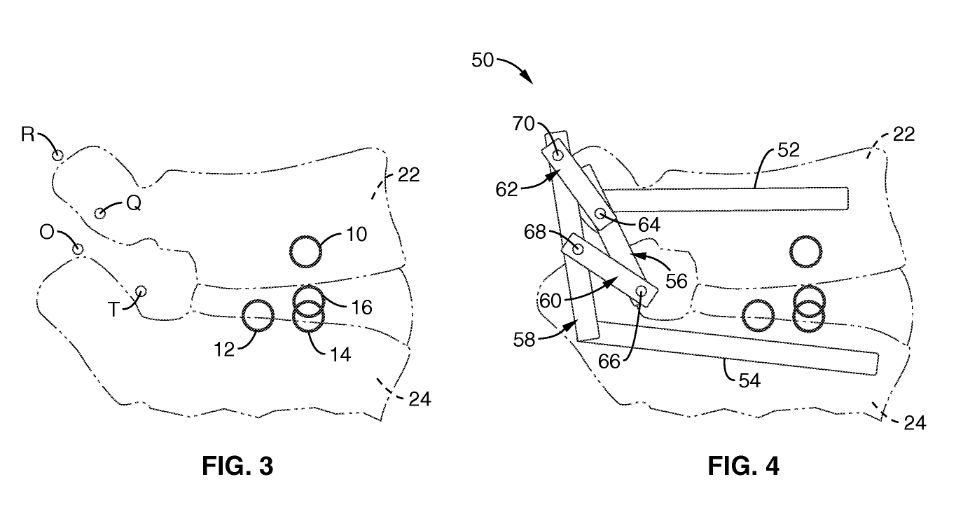

FIG. 3 shows an example of a calculated plot for a kinematically-defined linkage for flexion/extension by reference to the natural disc centroid gathered from experimental observation of the L5/S1 interspace, as elsewhere herein described in further detail. If points T and Q are defined as part of the L5 vertebra, then complementary points R and O are determined using the centrode locations 10, 12, 14, and 16 and points T and Q. Points R and O are fixed relative to S1. Points T, Q, R, and O were derived kinematic (graphical and/or computer generated) analysis of experimental test data described in further detail below.

The posterior linkage of the present invention, disclosed in further detail below, include links connecting points R-Q and O-T.

FIG. 4 shows a schematic lateral view of a posterior dynamic spinal stabilization system 50 in accordance with one embodiment of the present invention. The system 50 is adapted to closely approximate the centrode of natural kinematic linkage of the spinal joint (as detailed as points 10, 12, 14, and 16 in FIGS. 2A-2B and FIG. 3. The system 50 is defined mechanically via a four-bar kinematic linkage that bridges vertebral anchors (e.g. pedicle screw extensions or the like device known in the art), one pedicle screw 52 in each of the pedicles of the L5 vertebra 22, and another screw 54 in each of the pedicles of the sacrum 24.

The geometry of the linkage is defined by vertebral geometry and the natural intervertebral centrode. The linkage is designed to balance the centrode defined for flexion/extension, lateral bending and axial rotation. As shown in FIG. 4, two pedicle screws 52, 54 (which may also comprise porous-coated rods or similar anchoring mechanism) are affixed to adjacent vertebrae (superior vertebra 22 and inferior vertebra 24). The pedicle screws 52, 54 may be installed with posterior access to the spine via methods commonly used in the art.

A superior extension member 56 is rigidly attached to the superior pedicle screw 52 and extends downward toward the inferior vertebra 24. An inferior extension member 58 is rigidly attached to the inferior pedicle screw 54 and extends upward toward the superior vertebra 22. Upper articulating link 62 and lower articulating link 60 rotatably connect the superior extension member 56 and inferior extension member 58 via flexible hinge joints 64, 66, 68, and 70. It should be noted that joints 64, 66, 68, and 70 correspond to the locations of T, Q, R and O of FIG. 2, which were derived from centroid locations.

Superior extension member 56 and superior pedicle screw 52 move as a unit with the superior vertebra 22. Correspondingly, inferior extension member 58 and inferior pedicle screw 54 move as a unit with the lover inferior vertebra 24.

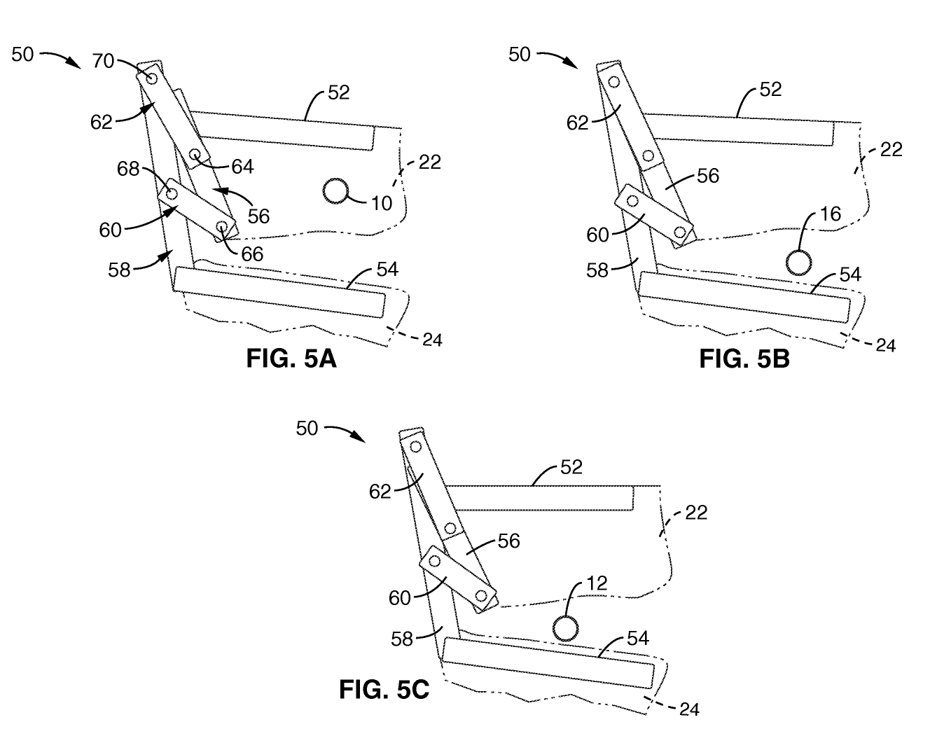

In order to provide a further overall understanding of the structural roles and operation of these various components in the overall assembly during use, FIGS. 5A through 5C show three modes of operation. As indicated across different ranges of motion, the IAR shifts according to use of this assembly in a manner that more closely approximates the natural centrode. In particular, the IAR shifts both laterally and vertically with respect to the spinal axis, and the dominant component of which depends upon angle and degree of motion.

FIG. 5A shows the spinal stabilization system 50 accommodating the position of the superior vertebra 22 at 6 degrees of flexion with the linkage assembly guiding rotation about the appropriate IAR 10.

FIG. 5B shows the spinal stabilization system 50 accommodating the position of the superior vertebra 22 at a neutral flexion position with the linkage assembly guiding rotation about the appropriate IAR 16.

FIG. 5C shows the spinal stabilization system 50 accommodating the position of the superior vertebra 22 at 6 degrees of extension with the linkage assembly guiding rotation about the appropriate IAR 12.

It is to be appreciated that the spinal stabilization system 50 shown in FIGS. 4 and 5A-C may be provided and used as a stand-alone dynamic fusion device. Alternatively, the assembly 50 may be used in conjunction with nucleus replacement or disc biologic regeneration strategies.

It is appreciated that the present embodiment shown in FIGS. 4 and 5A-C is directed primarily toward the L5/S1 joint of the spine. However, it is contemplated that the techniques and systems of the present invention may be used to stabilize a number of other areas of the spine, including L1/L2, L2/L3, L3/L4, L4/L5, and other vertebrae in the thoracic and cervical spine.

It is also appreciated that a number of mechanical variations can be implemented instead of or in combination with the hinge joints at points T, Q, R, and O shown in FIGS. 4 and 5A-C. An elastic or deformable material may be used that allows and restrains motion in the same path as provided by the hinged joints. For example, portions of the extension members 56, 58 and articulating links 60, 62 may be relieved (or coupled with an elastic material) at the specified locations to have a smaller cross-section to allow bending at the specified location (e.g. at points T, Q, R and O). The deformable material may comprise a memory material, such as nitinol, or a polymer having similar properties.

Various unique aspects of a system according to the various embodiments of the present invention include, without limitation, the following.

In one regard, the vertebrae are constrained sufficiently to facilitate distraction during surgical placement. In another regard, the linkage geometry of spinal stabilization system 50 guides the vertebra along the natural centrode. In still another aspect, the spinal stabilization system 50 can be placed via a posterior approach using minimally-invasive techniques. This is shown by way of further example in FIGS. 4 and 5A-C.

Previously disclosed devices intended to provide posterior dynamic stabilization generally either constrain movement about a fixed axis of rotation, or a variable axis that is far from the natural centrode. It is to be appreciated that the present invention responds to recent experimental data and observations providing more insight as to the natural centrode, and provides the appropriate systems and methods to more closely approximate this natural motion.

It is also to be appreciated according to the present embodiment shown in FIGS. 4 and 5A-C that the lower and upper articulating links 60, 62 of spinal stabilization system 50 can be locked (or at least provide a lockable option) so that the spinal stabilization system 50 may be employed to create a solid fusion as with standard fusion hardware. This may provide a benefit in certain particular circumstances for certain patients.

It is also to be appreciated that the spinal stabilization system 50 shown in FIGS. 4 and 5A-C is adapted for posterior dynamic stabilization of the L5/S1 spinal joint shown. However, the spinal stabilization system 50 may be employed to provide similar beneficial results to also more closely approximate the centrode of IAR movement in the normal spine versus prior attempts. For example, anterior or lateral dynamic stabilization assemblies and related implant methods may be adapted for use in treating patients according to the information provided herein without departing from the intended broad scope of the various aspects of the present invention. Moreover, engineered combinations of individual implants working together for an overall result may be used. For example, a posterior dynamic stabilization assembly may be provided in combination with at least one other implant, such as for example a disc implant (either nucleus or whole disc implant), or for example with an anterior or lateral dynamic stabilization implant, such that the overall assembly working together provides the desired range of motion about a more physiologic centrode of disc rotation.

Notwithstanding the foregoing, however, the particular approach of posterior dynamic stabilization as herein described by way of the particular embodiments is nonetheless considered of particular unique benefit and especially well adoptable mode of use in many medical procedures.

Other suitable modifications may also be made to the present particular exemplary embodiments in order to accommodate other joints or anatomical variations, with results consistent with the broad aspects of the present invention. Such anatomical variances may include, for example, different considerations at different spine levels, or patient-to-patient variances of anatomy at similar levels along the spine. For example, different sizes, angles, and relative placements of the component parts of the assembly shown may be made available to accommodate such variances. In this regard, further experiments may be conducted similar to those described herein, or appropriately modified by one of ordinary skill based upon an informed review of this disclosure and other available information, to suitably characterize the normal spinal motion across such variable parameters. Such experimental observations may then be used to form additional assemblies and methods that are adapted to suitably operate in a manner that approximates the normal motion according to the particular anatomical parameters characterized.

Information regarding other attempts to which one or more aspects of the present invention is intended to improve includes reference to one or more of the following, by reference: Dynesys from Zimmer Spine; and Isobar TTL from Scient'x USA.

The following more detailed description provides a more detailed understanding the various broad aspects contemplated hereunder, and furthermore conveys additional highly beneficial embodiments shown and described.

### Experiment

The following description relates to certain highly beneficial embodiments that insightfully respond to the experimental design, results, and observations that the instant axis of rotation of spinal joints influences facet forces during flexion/extension and lateral bending. This is described by way of particular example at L5/S1 joint, a prevalent location for compromised spinal joint dynamics and medical surgical intervention.

Because the disc and facets work together to constrain spinal kinematics, changes in the instant axis of rotation associated with disc degeneration or disc replacement may adversely influence risk for facet overloading and arthritis. The relationships between L5/S1 segmental kinematics and facet forces are not well defined, since previous studies have separated investigations of spinal motion and facet force. The goal of this cadaveric biomechanical study was to report and correlate a measure of intervertebral kinematics (the centrode, or the path of the instant axis of rotation) and the facet forces at the L5/S1 motion segment while under a physiologic combination of compression and anterior shear loading.

Twelve fresh-frozen human cadaveric L5/S1 joints (age range 50 to 64 years) were tested biomechanically under semi-constrained conditions by applying compression plus shear forces in several postures: neutral, and 3 degrees and 6 degrees flexion, extension and lateral bending. The experimental boundary conditions imposed compression and shear representative of in vivo conditions during upright stance. The 3-D instantaneous axis of rotation (IAR) was calculated between two consecutive postures. The facet joint force was simultaneously measured using thin-film sensors placed between both facet surfaces. Variations of IAR location and facet force during motion were analyzed.

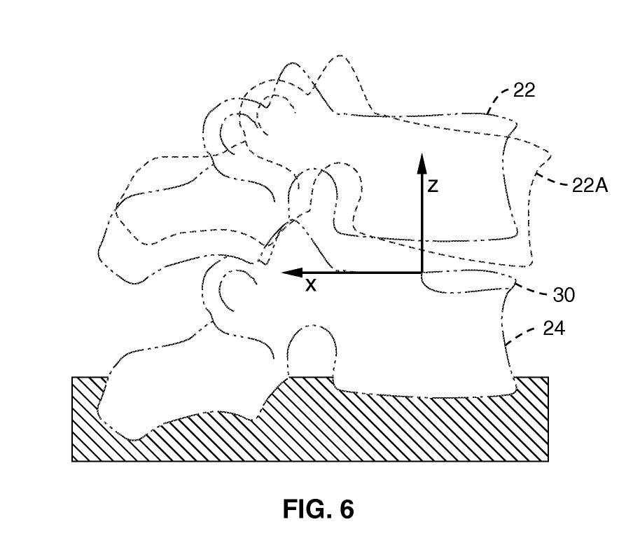

FIG. 6 illustrates an exemplary model of an L5/S1 joint and the respective coordinate system used in this experiment. As shown the x-z plane is shown parallel to the sagittal plane, with the y-axis pointed vertically. The origin 30 is the center of the superior endplate 26 of S1 vertebra 24.

During flexion and extension, the IAR was oriented laterally. The IAR intersection with the mid-sagittal plane moved cephalad relative to S1 endplate 24 during flexion (p=0.010), and posterior during extension (p=0.001). The facet force did not correlate with posture (p=0.844). However, changes in the facet force between postures did correlate with IAR position: higher IAR's during flexion correlated with lower facet forces and vice versa (p=0.04). During lateral bending, the IAR was oblique relative to the main plane of motion and translated parallel to S1 endplate 26, toward the side of the bending. Overall, the facet force was increased on the ipsilateral side of bending (p=0.002).

The IAR positions demonstrate that the L5 vertebral body 22 primarily rotates forward during flexion (IAR close to vertebral body center) and rotates/translates backward during extension (IAR at or below the L5/S1 intervertebral disc). In lateral bending, the IAR obliquity demonstrated coupling with axial torsion due to resistance of the ipsilateral facet.

Accordingly, the present experiment simultaneously measures spinal kinematics and facet forces during motion in a human cadaveric model of the healthy L5/S1 joint under physiologic compression and shear. This provides a new insight into the centroid of spinal motion, to which the present system and method embodiments variously relate with novel solutions.

The lumbosacral spine was harvested from 12 human donors aged 50 to 64 at the time of death (8 male and 4 female). Only specimens with no radiographic evidence of bone disease or joint degeneration (osteophytes, disc space narrowing, facet hyperthrophy) were used in this study. Specimen preparation consisted in meticulous removal of muscular tissue so as to retain the integrity of the capsular and ligamentous elements. For each specimen, the superior half of the L5 vertebra and inferior half of S1 vertebra were potted in polymethylmethacrylate (PMMA), so that S1 end-plate was parallel to the PMMA surface and clamping faces.

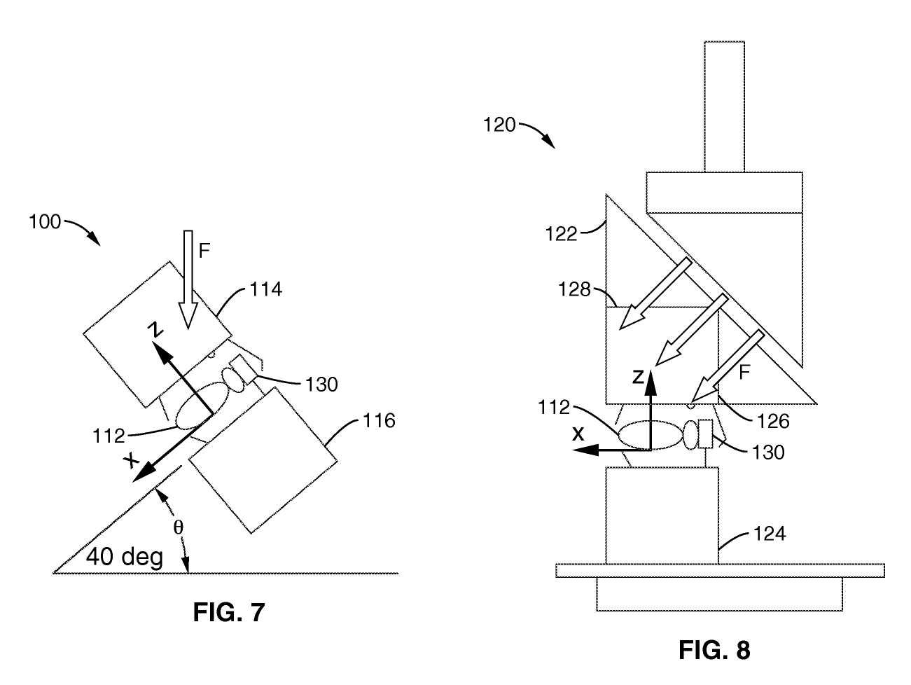

Each specimen was placed in a servo-hydraulic apparatus (e.g. Bionix 858, MTS Systems Corp. Eden Meadow, Minn.) such that the disc was oriented at 40 degrees relative to the horizontal axis. FIG. 7 shows a schematic diagram of L5/S1 testing assembly 100 in this arrangement, per 40° sacral slope and 850 N load in standing position. FIG. 8 illustrates a testing assembly 120, which constrains the L5 posture in flexion, extension, and bending for investigating L5/S1 kinematics. The applied load N is uniformly distributed and applied in both shear and compression. Axial torsion was unconstrained. The angle θ was chosen to reflect the average 39 degrees sacral slope in standing position.

Referring to FIG. 7, the specimens 112 were loaded with an 850 N vertical force applied via near frictionless elements 112 and 114 (e.g. polished steel lubricated with machine oil). The force N was chosen to match estimates for L5/S1 in the standing position based on disc pressure and myo-electric measurements, and therefore represents both gravity and muscular loading. The 850 N vertical force generated 650 N of disc compression 126 and 550 N of horizontal shear 128 consistent with free body analyses of L5/S1 based on specific morphometric studies. The semi-constrained feature of the testing apparatus 110 is such that the location of the resultant force at the frictionless surface varies, and thereby minimizes its distance to the IAR. Consequently, confounding moments about the IAR are minimized. In addition, at the start of each experiment, the rotational actuator of the test system was used to adjust the axial rotation position of the frictionless surface so as to minimize differences in bilateral facet forces. This adjustment procedure accounted for any slight misalignment of the L5/S1 specimens within the PMMA.

Wedges were added at the frictionless interface to impose 3 and 6 degrees flexion/extension and lateral bending postures. The 12° total range of motion in the sagittal and the frontal plane was below the normal physiological zone of the L5/S1 joint. Automatic coupled torsions were allowed in the oblique frictionless plane. Each 3° rotation between 2 consecutive postures defined a ‘motion sector.’

Specimen preconditioning consisted in 10 cycles of complete loading and unloading in neutral posture over 5 minutes. During testing, data were collected after 2 minutes of loading for each posture. Tissues were kept moist during testing by wrapping in saline-soaked gauze.

Outcome Measures

a. Instantaneous Axis of Rotation (IAR)

For a rigid body in three-dimensional (3-D) space, the motion from one position to another can be described by the sum of a rotation around a single axis and a translation (perpendicular to the plane of rotation) along this axis. For that general case, the axis is called the helicoidal axis. For small displacements, movement occurs around an ‘instantaneous axis.’ For a null translation along the axis, the instantaneous helicoidal axis is called instantaneous axis of rotation (IAR). All necessary information to calculate the instantaneous helicoidal axis are contained in the transformation matrix, which is a mathematical description of the rigid-body movement from one position to another, and includes a square 3×3 rotation matrix and a 3×1 translation matrix. Consequently, the helicoidal axis is just an alternate representation of the transformation matrix.

The transformation matrix was calculated using the method of Kinzel, based on 3-D coordinates of four non-coplanar landmarks placed on the moving vertebra (L5). Then the direction and the position and of the axis was determined in the 3-D space according to the method of Spoor and Velpaus. Finally these data were transformed to a local coordinate frame based on the radiographic anatomy. The origin of the orthogonal right-handed frame was the center of the endplate of S1 24, the X-axis being sagittal, the Y-axis coronal, and the Z-axis vertical and perpendicular to the endplate (see FIG. 6). IAR direction was described using the inclination (angle θ between the axis and the horizontal plane that is equivalent to a latitude from the S1 endplate) and the declination (D; angle between the axis and the sagittal plane that is equivalent to a longitude). IAR position was described as the position of the unique point, P (xₚ, yₚ, zₚ), of the axis so that the distance OP is the shortest distance from the origin (O) to the axis (P). Therefore, OP is perpendicular to the IAR.

Using the direct linear transformation method, three Falcon strobe cameras, utilizing Eva 6.0 software (Motion Analysis Corp. Santa Rosa, Calif.) established the 3-D coordinates of four reflective markers placed on L5, and of one reflective marker a fixed on S1 for each posture of L5. The transformation matrix and IAR between consecutive postures of L5 were computed using the average of 300 repeated measures of the position of each marker collected at each posture. The marker on S1 was also visible on specimen radiograph for matching the IAR to the specific anatomy of each specimen.

Despite the precision of the strobe cameras for determining the markers coordinates (±0.25 mm), random error was propagated and magnified by the algorithm of matrix computation. Woltring assessed that the error in IAR position was inversely proportional to the amount of rotation: random error tends towards infinity when rotation tends towards zero. Other factors like the marker distance to the real IAR, and the radius of distribution of the markers, also can contribute to the error magnitude. A standard door hinge oriented in a pre-defined direction was utilized to determine the accuracy of the experimental set up in the IAR calculation. Based on a pilot study using this approach, the estimated absolute error for IAR placement during pure rotation: 3° movements as 4 mm, and IAR direction as 1°.

b. Facet Force

Simultaneous to the IAR calculation, the compression force transmitted through the left and right facet joints was recorded using thin pressure sensors 130 (e.g. Flexiforce A101-500, Tekscan Inc, South Boston, Mass.). The sensors 130 were introduced into the right and left joint space through a vertical cut in the joint capsule. The sensors 130 were 10 mm in diameter, 0.2 mm thick, and made of flexible mylar and contain ink whose resistance varies linearly to the applied force. Sensor output was recorded at 5 Hz and averaged using data acquisition software (Labview 6.1, National Instruments, Austin, Tex.). The sensor 130 was calibrated by applying pre-determined forces via contact surfaces of different areas and demonstrated that the output voltage varied linearly with the force regardless of the pressure area. The calibration ratio was 500 N/V (±5%).

For a given rotation, it is assumed that the facet force variation (difference in facet force between adjacent postures, δF) would be proportional to the distance (d) between IAR and facet joint,

δF∝d Eq. 1

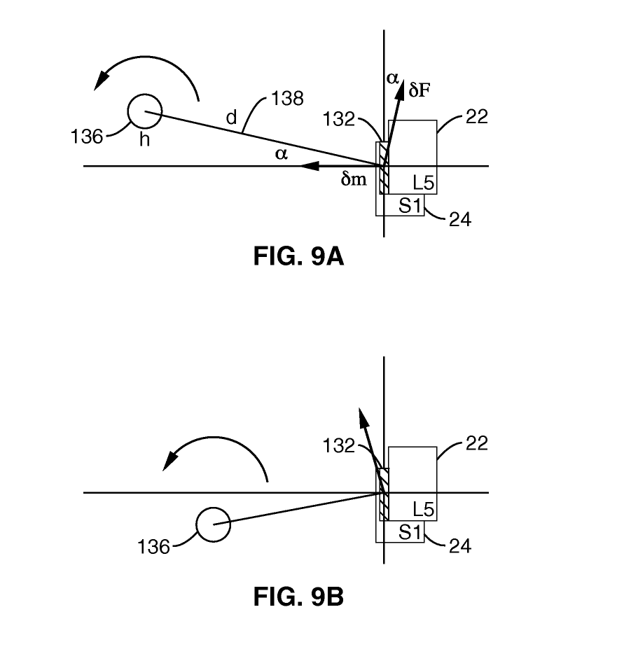

However, the force sensor introduced in the joint records only the force component perpendicular to the joint surface (δm). If α is the angle between the sensor surface and δF (as shown in FIG. 9A), then

δm=δF·sin α Eq. 2

By combining equations (1) and (2), one gets,

δm∝d·sin α Eq. 3

As the facet joint space 132 is considered vertical (orthogonal to the superior S1 endplate 26), α corresponds to the angle between the endplate and the line 138 between the facet joint and the IAR 136. Then

d·sin α=h Eq. 4

where h is the IAR height relative to the facet joint level.

Consequently, from Equations 3 and 4, it is apparent that the facet force is proportional to the IAR height,

δm∝h Eq. 5

To test this hypothesis, changes in the facet force measurement were compared between adjacent postures (δm) to the calculated IAR 136 height h for those adjacent postures.

All statistical analyses were performed using SPSS statistical software (Version 11.5, SPSS Inc., Chicago, Ill.). Standard analysis of variance (ANOVA) procedures were used to compare group means and to estimate the effect of the specimen variables (parent specimen, motion sector, and direction of motion, entered as categorical variables) on the measured parameters of interest (IAR position and direction, and facet force, entered as continuous variables). When appropriate (P<0.05), LSD post hoc tests were performed to identify group subsets with significant differences. Left and right facet forces were combined so that they were considered repeated measures within the same specimen in flexion/extension. In lateral bending, they were combined relative to the bending direction.

Since the facet force was measured with a single sensor 130 that is 10 mm in diameter, the specific location of the contact force could not be distinguished within that zone. Therefore, the facet force variation (δm) for those postures where the IAR 136 was above the facet sensor zone was compared against those postures where the IAR was below the facet sensor zone using one way ANOVA.

Flexion/Extension

In flexion/extension, the IAR 136 direction was similar for every motion sector. The average inclination was 1.3° (p=0.37) and the average declination was 91.4° (p=0.701). The IAR was therefore considered substantially perpendicular to the main plane of motion, and its position was described as its intersection with the mid-sagittal plane (yᵢ=0, see FIG. 1A and Table I).

The x-coordinate of the IAR intersection with the mid-sagittal plane (xᵢ) was significantly different between motion sectors (p=0.001). Post-hoc tests showed that the IAR was more posterior for the motion sector between 3° and 6° extension than for all other sectors. The z-coordinate of the IAR intersection with the midsagittal plane (zᵢ) was significantly different between motion sectors (p=0.010). Post-hoc tests showed that the IAR was significantly higher between 3° and 6° flexion than for all other sectors.

Referring back to FIGS. 1A and 1B, intersections of the IAR 136 and the sagittal plane 18 and the coronal plane through the center of the disc are represented on a lateral FIG. 1A and an AP radiograph FIG. 1B respectively. The diameter of the circles corresponds to the average error in position (4 mm). The circle numbering 10, 12, 14, and 16 represents differing positions from extension to flexion and from left to right lateral bending

As illustrated in Table II, the facet force did not vary consistently with posture during flexion/extension (p=0.844).

However, during flexion movements, the facet force variation (δm) was significantly less when the IAR was above the facet sensor zone as compared to when it was below this zone (p=0.04; FIG. 6). The average transmitted force through each facet for all specimens was 49.5 N.

In sum, the IAR, during flexion and extension, is oriented laterally. The IAR intersection with the mid-sagittal plane 18 moves cephalad relative to S1 endplate 26 during flexion, and posterior during extension. The IAR positions demonstrate that the L5 vertebral body 22 primarily rotates forward during flexion (IAR close to vertebral body center) and rotates/translates backward during extension (IAR at or below the L5/S1 intervertebral disc).

Lateral Bending

Table III illustrates the coordinates of the IAR position in lateral bending, and the IAR declination was 1.80 and similar for every motion sector (p=0.565). The IAR inclination varied with the motion sector (p=0.011); post hoc test demonstrated that it was significantly higher after 3° in left lateral bending, and changed in right lateral bending. Because of the IAR obliquity, the IAR position was described in the general case as the position of P (see FIG. 1B).

The x-coordinate of the IAR position (xₚ) varied with the sector of motion (p=0.002). Post hoc testing showed that the IAR was more posterior beyond 3° bending in both directions. The y-coordinate of IAR position (yₚ) varied significantly according the sector of motion during lateral bending (p<0.001). Post-hoc tests showed that the IAR moved horizontally towards the bending beyond 3° bending in both directions. The z-coordinate (zₚ) varied between sectors of motion (p=0.036). Post hoc tests showed that the IAR was higher beyond 3° lateral bending in both directions.

Table IV shows the facet force in lateral bending. The facet force was related to the posture (p=0.002) in lateral bending and increased to the side of the bending. Post-hoc tests demonstrated that facet force increased significantly in the first 3° lateral bending.

In sum, the IAR, during lateral bending, is oblique relative to the main plane of motion and translates parallel to S1 endplate, toward the side of the bending (Table II).

In lateral bending, the IAR obliquity demonstrates coupling with axial torsion due to resistance of the ipsilateral facet.

The study investigated various relationships between intervertebral kinematics and facet forces during physiologic motion and loading of the L5/S1 joint. The observed IAR was normally located in the posterior part of the intervertebral disc, and moved superiorly during flexion, posteriorly during extension, and ipsilaterally during lateral bending. As expected, coupled axial rotation was associated with lateral bending. While the facet force did not show a uniform variation in flexion/extension because of interspecimen variability, it was correlated with the horizontal IAR displacement in lateral bending, such that the facet force increased in the ipsilateral facet.

The observed IAR was perpendicular to the sagittal plane in flexion/extension and located at the posterior part of the intervertebral disc, which is consistent with prior reports based on planar measurements in vitro and in vivo using the graphical method of Reulaux. The Reulaux method calculates the instantaneous center of rotation by drawing bisectors between landmarks on successive radiographs or photographs. This 2D method is less accurate compared to the 3D approach used in the current study, which may explain why various of the current observations—including for example but without limitation that the IAR moves superiorly, perpendicular to S1 endplate during flexion, and posteriorly, parallel to the endplate during extension—have not been described previously.

The IAR path relative to S1 endplate 26 demonstrates that from extension to flexion, the L5 vertebra 22 primarily translates anteriorly at first (i.e., the IAR is low during motion between 6 and 0 degrees of extension), and subsequently rotates forward when at the flexion limit (since the IAR approaches the geometric center of L5 during motion between 3 degrees and 6 degrees of flexion). This motion in flexion/extension reflects posture-varying roles of the disc and facet joints in constraining movement, and is consistent with reports that the facet contact area moves upwards into flexion.

Significant interspecimen variability was observed in the facet force trend with posture that is contrary to the classical notion that facet forces systematically increase into extension. This discrepancy may be due at least in part to different loading conditions—prior studies were conducted using either pure moments or axial compression while we utilized compression plus anterior shear. Additionally, this data is believed to be the first to demonstrate a significant vertical IAR movement relative to the S1 endplate.

The vertical IAR motion is a likely factor in facet force variation during sagittal motion, since the facet joint space theoretically opens or closes depending on whether the IAR is above or below the level of the joint, as illustrated in FIGS. 9A and 9B.

FIG. 9A shows a schematic lateral view of the facet joint 132 of the L5/S1 vertebrae. Facet force variation 6F in the facet joints in flexion and extension is related to height H of the IAR 136, assuming that the L5/S1 facets are perpendicular to the S1 endplate 26. Facets open into flexion when the IAR 136 is above the facet level, as shown in FIG. 9A. Facets close into flexion when the IAR 136 is below the facet level as shown in FIG. 9B.

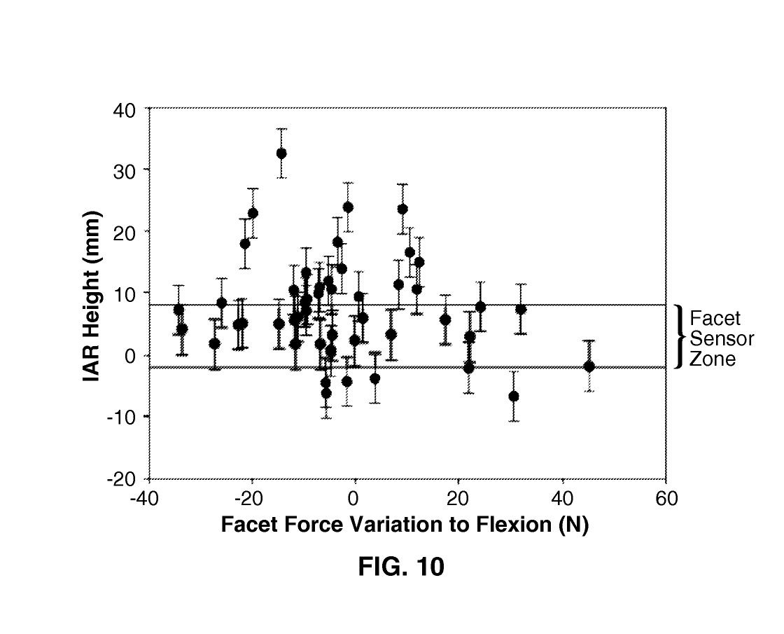

If the facet joint spaces 132 are considered vertical (i.e. perpendicular to the S1 endplate 26), the IAR height H and facet force should be related, and generally linearly, during flexion/extension. FIG. 10 shows a graph of IAR 136 distance to the S1 endplate 26 (zᵢ: mm) plotted against facet force variation δF (N) for each 3° rotation into flexion. The “grey zone” corresponds to the facet height and hence force sensor 130 location. The average facet force variation between two consecutive postures during flexion was −4.8 N when the IAR 136 was located above the force sensor 130, and +7.2 N when the IAR 136 was located below the force sensor 130 (p=0.040).

The IAR height H is related to the facet force variation in flexion/extension. Therefore, the present experimental data, as seen by reference to the graph in FIG. 10, demonstrates that the IAR height H determines whether the facets 132 open or close during sagittal plane movements (as further described for example by reference to FIGS. 9A and 9B).

The lateral bending experimental data revealed IAR 3-D obliquity, which is due to coupling between lateral bending and axial rotation. That is, if lateral bending were not associated with axial rotation, then the IAR direction would have been perpendicular to the plane of bending. Since bending was applied by simulating the 40° sacral obliquity (FIGS. 7 and 8), the expected IAR inclination would have been 40°. Rather, the actual average IAR inclination was 28.2°, with the 11.8° difference due to induced coupled rotation perpendicular to the main plane of motion. By decomposing the moment relative to the main plane of motion and its perpendicular plane (the frictionless surface in the testing device), the data demonstrates that the coupling was such that right bending of the L5 vertebra 22 was coupled with right axial rotation, and vice versa. This result, under semi-constrained shear and compression in the oblique lumbo-sacral joint, is consistent with the observation of others when documenting coupled rotations of L5/S1 under pure moment loading conditions in vitro and in vivo.

Horizontal IAR displacement to the side of the bending is contrary to previous reports of using 2-D data. This may be due to 3-D coupled motion as a confounding factor in previous 2-D methods. Because the simultaneous horizontal IAR pathway and facet force increase to the side of the motion (significant Pearson correlation, p=0.015), it is believed that the ipsilateral facet blocks L5 lateral translation in bending. The IAR inclination increases and posterior displacement beyond 3° bending confirm that the impingement of the ipsilateral facet leads to coupling between lateral bending and axial torsion.

These experimental results included testing boundary conditions that allowed 4 degrees of freedom for L5 (compression, AP translation, lateral translation, and axial torsion). Two degrees of freedom were constrained (sagittal and frontal rotation). The fact that the testing device was semi-constrained may have led to asymmetry in facets impingement, because of the inevitable slight misalignment of the specimen in the apparatus. Facet asymmetry may have introduced artifacts in the kinematic or facet data, in spite of the rotational pre-adjustment of the apparatus. However, the current methodology provides a major advantage by utilizing a uniform and controlled load on the superior vertebra that results in physiologic combinations of compression plus shear. As the IAR is unknown before testing and mobile during motion, other loading conditions that use a fixed axis force would have theoretically generated variable and unknown moments around the IAR leading to uncertain boundary conditions and uncertain results.

Algorithm and instrumentation factors limited the IAR precision to ±4 mm for 3 degrees of movement. Consequently, IAR movements less than this limit could not be detected reliably. Yet, despite this and inevitable specimen-to-specimen variability, several statistically significant trends in IAR position and facet force were readily observed.

The circular area of the force sensor 130 that was used was about half the size of the facet joint 132 surface. As a potential result of this mismatch, it is possible that the facet contact area may have moved beyond measurement area during testing. However, the relatively continuous nature of the facet force measurements between postures suggested that this was not the case. In addition, a vertical cut in the facet joint capsule was necessary for inserting the sensors during preparation. This did not appear to adversely affect segmental kinematics as has been reported by others using pressure Fujifilm paper for mapping facet forces.

The experimental results demonstrate consistent relationships between IAR location and facet forces. These relationships highlight the interaction between the intervertebral disc and posterior elements for both load support and kinematics constraint. It is believed that the specific location of the IAR during motion and its influence on facet joints may be related to the initiation of facet arthritis. For example, since it has been suggested that erratic IAR locations are associated with degenerative disc disease, the present data suggest that these non-physiologic IAR locations may, in turn, increase facet forces and subsequent arthritis risk.

The relationships between IAR location and facet force during compression and shear loading are used to disc arthroplasty and other medical therapeutic devices of the present invention to more accurately affect spinal motion and integrity.

The therapeutic system and method of the present embodiments combine disc replacement and facet joint modification to provide a highly beneficial and improved result versus disc replacement alone. Through a combination of joint distraction (during device implantation) and IAR optimization, the device therapy regimen according to certain aspects of the present invention is adapted to reduce facet forces and thereby protect the joints from iatrogenic arthritis. The present data supports the conclusion that the system of the present invention provides a highly beneficial and improved interventional system and method by maintaining an IAR path that is cephalad during flexion, posterior or caudal during extension, and lateral in bending. Such a result more closely approximates the experimentally observed kinematics of the intact L5/S1 level.

Motion is complex due to position-dependent interaction between disc and facets—during axial rotation, lateral bending, or extension the facets become more engaged (have higher forces) than during flexion. Consequently, during axial rotation, lateral bending, and extension, the IAR moves toward the facet joints. Implants that are meant to facilitate intervertebral motion may conflict with normal motion patterns, and when this occurs it will cause higher than normal force generation in either the facets or discs. Defined, three-dimensional patterns of normal intervertebral motion can therefore serve as a basis for design of dynamic stabilization devices so that the device-constrained motion can more closely match normal and thereby keep tissue stresses (and risk for back pain) minimized.

The normal motion can be parameterized using the Instant-axis-of-rotation which is a line is space that an object rotates about and translates along as it moves from one position to another. The IAR is analogous to the path of a thrown football—the ball is rotating about and traveling along the path.

Dynamic stabilization in the context of spinal implant of the present invention can be defined as constrained intervertebral movement, where the constraint is meant to eliminate unwanted, non-physiologic motions. The premise is that non-physiologic motion patterns are painful by creating elevated stresses in the disc and facets. Dynamic stabilization devices generally contact and guide adjacent vertebral movement. Two spaces are generally targeted for dynamic stabilization devices to reside (e.g. where they don't conflict with important structures such as neural or vascular elements). These are posteriorly in the region of the erector spinae muscles, or anteriorly in the intervertebral disc space. Posterior devices are generally attached to vertebra by pedicle screws. Intervertebral devices typically attach via metal endplates. The particular embodiments described here for physiologic dynamic stabilization (PDS) are exemplary of the highly beneficial posterior approach, but can be accomplished for example by a family of posterior devices and intervertebral devices that will be described elsewhere hereunder.

Posterior

As elsewhere herein described by way of particular embodiments, a family of devices may be provided that attach via pedicle screws or pedicle devices. Instrumentation related to such approach may include linked, hinged, deforming, or sliding members that, working together, facilitate intervertebral motion as herein described.

Intervertebral

A family of intervertebral devices in accordance with the present invention may include linked, hinged, deforming, or sliding members that work together to facilitate intervertebral motion as described herein. Due to space constraints, the device may include metal endplates with contoured articulating surfaces (which may be, for example, similar in certain regards to previously disclosed ‘kinematic’ knee replacements). The contoured surfaces, according to one embodiment, have position-dependent contact points with orientations of the mating surfaces such that, as the vertebra rotates, the surface constraint guides the proper kinematics in all three planes of motion.

Further embodiments of the present disclosure are described below, each providing certain particular contemplated benefits considered unique. In addition, certain of the broad aspects and other modes or embodiments are further exemplified by certain such additional specific features and embodiments.

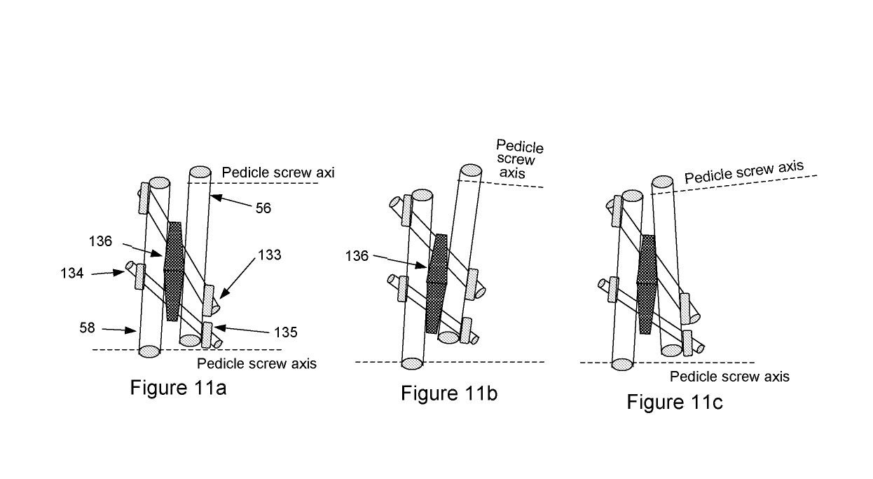

FIGS. 11a-c show one particular further embodiment in various modes of use as follows. Two members 56 and 58 that are connected or otherwise coupled to pedicle screws 52 and 54 respectively. Member 56 is connected to the superior vertebra and is anterior to member 58. Members 56 and 58 are connected together by two pliant straps 133 and 134 that may be composed for example of solid or woven polymer, or may be rubber based, composite, etc., to suit a particular purpose. The pliant straps 133 and 134 pass through or around extension members 56 and 58 and are secured by clips 135 or other forms of securing devices. The orientations of the pliant straps 133 and 134 (relative to the members 56 and 58 and the anatomical axis of the spine) are the same or similar to the orientation of articulating link 62 and articulating link 60 as shown in FIG. 4. Members 56 and 58 are separated by a pliant spacer 136 that incorporates a taper at either end. Tension in the straps 133 and 134 is maintained by a spacing between members 56 and 58 that is defined by the pliant spacer 136.

In the specific embodiment shown, the spacer 136 includes about a 6-degree taper at either of its end superiorly and inferiorly oriented ends. More specifically, each tapered end portion includes two opposite tapered surfaces that converge toward the respective end. Each of these surfaces tapers by about 3 degrees, such that the overall taper between them accounts for about 6-degrees. This result is considered to correlate to a particular range of motion considered beneficial in the assembly for intended results in-vivo. The bar/strap/spacer construct constrains the spine unit to a physiologic motion pattern while the taper feature of the spacer limits the overall range of motion.

For further illustration, FIG. 11b shows the mechanism at flexion, and FIG. 11c shows the mechanism at extension. In these relative deflection states from “normal” or resting, the relative motion between members 56,58 confront the tapered surface of the spacer 136 at the extent of respective ranges of motion. While further motion may be possible, resistance due to confronting engagement of the respective member(s) with the tapered surface of spacer 136 provides limited freedom beyond the angle of that engagement due to compression of spacer 154 to be experienced in that extended range of motion. In this regard, spacer 154 may be relative non-compressible under the anticipated forces of over flexion or over-extension beyond the limited range prior to confronting engagement with the respective member surfaces—this would more abruptly limit motion by the range of the tapered angles of spacer 154. Alternatively, spacer 154 may be characterized with some degree of compressibility under such anticipated forces, such that some further motion may be provided though under more significant constraining forces provided by the compression of that spacer 154 (and thus by the overall assembly on the spinal joint).

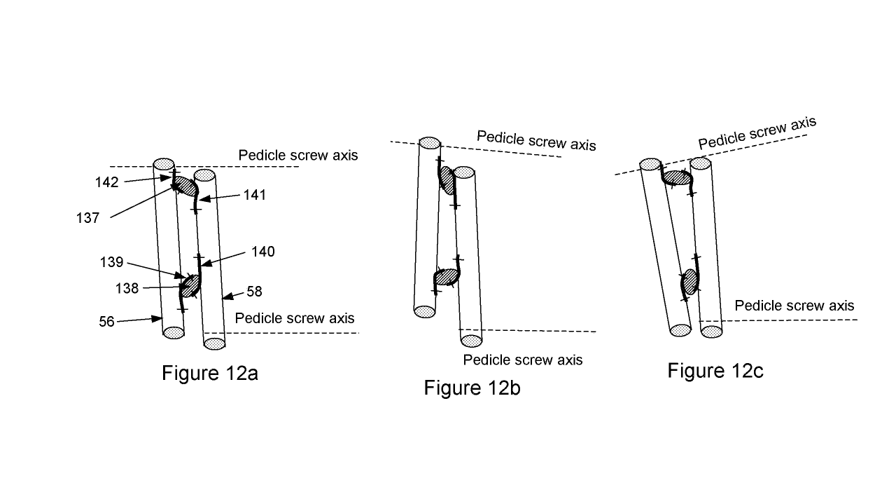

FIG. 12 shows a further embodiment that incorporates a cam-based assembly as follows. Two members 56 and 58 are connected to pedicle screws 52 and 54, respectively. Member 58 is connected to the inferior vertebra and is anterior to member 56. Cams 137 and 138 are provided in particular manner so as to articulate with members 56 and 58 via tracks, guides or groves in members 56 and 58. Cams 137 and 138 may also interact with and are connected to members 56 and 58 with arms or straps 139,140,141,142 such that the cams 137 and 138 roll and stay in contact with the members 56 and 58. The straps are provided in one highly beneficial further embodiment of shape memory or superelastic alloy construction, such as Nickel-Titanium alloy. Each strap has one end attached to the adjacent respective member 56 or 58, while the other end is attached to the adjacent respective cam, as shown. The straps guide rolling of the cams and provide appropriate stiffness under applied forces on the assembly to limit the range of motion of the spinal unit. That is, as the spinal unit moves away from the neutral position (FIG. 12a) the straps provide progressively-increasing stiffness that mimics the natural soft tissue constraints. The shape of cams 137,138 are such that they constrain the same or similar relative translation and rotation of members 56, 58 as do the linkages shown in other embodiments hereunder, such as for example links 60, 62 in FIG. 5. Accordingly, closely approximating the intended physiologic motion of the spine at the area of implant is achieved. For further illustration, FIG. 12b shows the mechanism at flexion, while FIG. 12c shows the mechanism at extension.

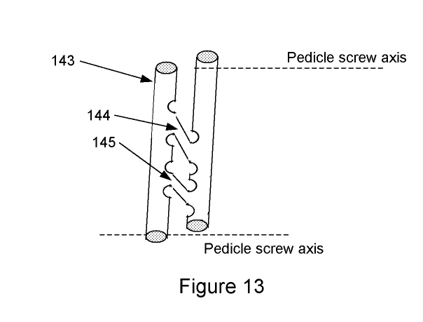

FIG. 13 shows another embodiment as follows. Body 143 is manufactured as one solid integrated unit, such as in one particular beneficial further embodiment from a solid shape memory alloy or polymer. Body 143 is configured in a manner such that relative vertebral body movement is facilitated via localized deformation experienced along various portions or component sub-parts of body 143. An internal portion of body 143 is manufactured into elements 144,145 that are in the same or similar relative orientation and length of links 62 and 60 of FIG. 5. The geometry of body 143 is such that relative movement of the attached vertebral bodies is the same or similar as that guided by the mechanism in FIG. 5.

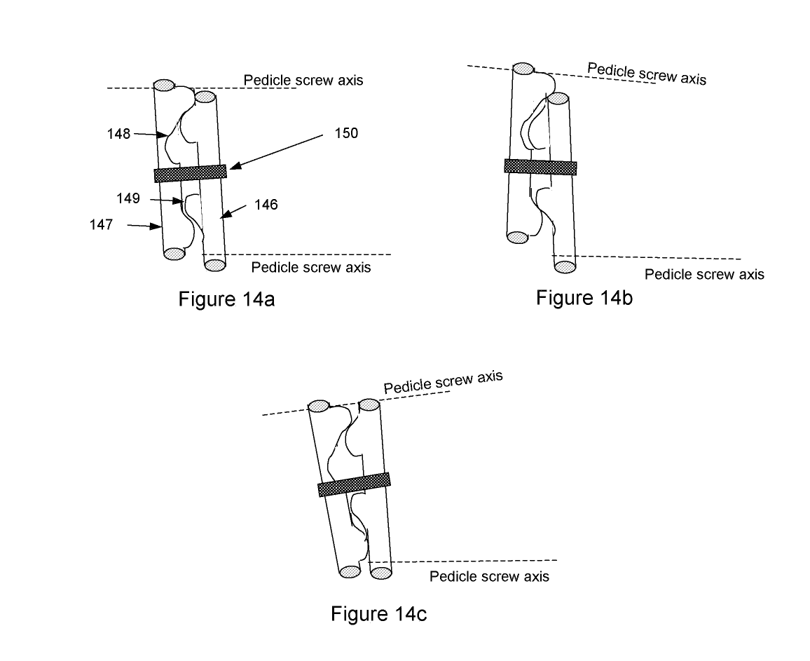

FIG. 14 shows another embodiment as follows. Two shaped members 146,147 are connected to inferiorly and superiorly positioned pedicle screws, respectively. Shaped members 146,147 interact via cam surfaces 148,149 that constrain the relative sliding and rotation of shaped members 147 and 148. Shaped members 146 and 147 are held together by spinal forces and band or strap 150, which may be in certain exemplary embodiments of elastomeric or compliant construction, and may be constructed of similar materials as provided for binding straps of other embodiments herein described or otherwise apparent to one of ordinary skill. The geometry of mating surfaces 148 and 149 are configured so as to guide the same or similar relative intervertebral movement of the assembly, and thus the spinal segment where the assembly is implanted, as mechanism in FIG. 5.

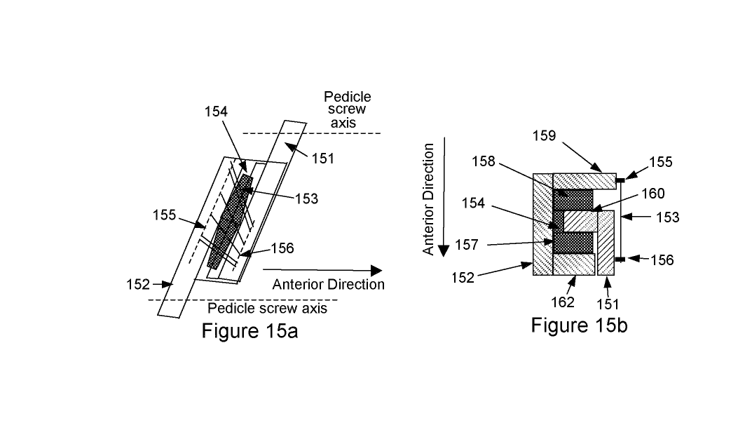

FIGS. 15a-b show certain aspects of another embodiment as follows.

As shown in FIG. 15a, members 151,152 are rigidly connected to pedicle screws 52, 54, respectively. Members 151,152 are separated by a spacer 154 that guides translation and rotation of members 151,152 while constraining their separation. Spacer 154 is of elastomeric construction in one highly beneficial particular embodiment. Members 151,152 are also connected via a multiplicity of tethers, such as shown for illustration at tether 153, that are attached to members 151,152 via a row of pins 156,155 respectively. Such tether(s) 153 is of relatively thin thickness in the highly beneficial particular embodiment shown, whereas the particular geometry or shape of any one or all of the tethers may be adapted as necessary to accomplish the overall objective within the context of the more specific details chosen for other features of the overall assembly.

The orientation of tethers 153, in the particular highly beneficial embodiment shown, varies between them. Such orientation in the illustrative embodiment shown is substantially parallel to that orientation previously presented by articulating link 60 shown in FIG. 5 at the superior end of the respective assemblies, and substantially parallel to that orientation previously presented by articulating link 62 also shown in FIG. 5 at the inferior end. Intervening thin tethers are oriented over a rotating range between these two specifically identified orientations. While this particular arrangement is considered of particular benefit, it is to be appreciated that the use of one or more tethers in the assembly may be varied in combination with other features to substantially provide the intended results herein contemplated and without departing from the intended scope of the present aspects broadly considered.

For further illustration, FIG. 15b shows a superior-inferior view of the details of how members 151,152 interact with spacer 154 and thin tethers 153. A portion 158 of spacer 154 is located between a lateral projection 159 of member 152 and a lateral projection 160 of member 151. This spacer portion 158 prevents posterior movement of bar 151 relative to bar 152. Similarly, another spacer portion 157 is located between the lateral projection 160 of member 151 and lateral projection 162 of member 152. This spacer portion 162 prevents anterior movement of bar 151 relative to bar 152. The combined actions of spacer 154 and thin tethers 153 act to guide the natural intervertebral movement in a similar manner as with the mechanism in FIG. 5.

It is to be appreciated by one of ordinary skill based upon a review of the entirety of this disclosure that, while certain particular embodiments are herein shown and described, various aspects are contemplated broadly and not to be considered limited by requiring only such specific embodiments. In one particular example, the present disclosure describes certain specific dynamic spinal stabilization assemblies which are configured in a particular way to provide a shifting IAR of spinal motion over different ranges of that motion. More specifically, such assemblies are adapted to move with a shifting IAR in two different directions/locations from resting position, thus corresponding with differences in IAR over certain ranges of spinal motion around “normal” (or resting), flexion, and extension motion. Still more specifically, a first general IAR location is associated with one range of spinal motion around “normal”. This shifts in one direction toward a second IAR point for flexion motion beyond that range around “normal”. The general IAR position corresponding with “normal” or resting motion range furthermore shifts in a second direction toward still a third IAR point—different from both the first and second IARs—for extension motion beyond that range around “normal”.

It is still further to be appreciated that such motion ranges described that correspond with about 3-degrees of rotation in either direction around normal corresponds generally with the first general IAR coordinates. Further motion beyond that “normal” or resting range, by up to about 3 further degrees in either direction, corresponds with the IAR shifts in first and second different directions from normal and toward the second and third still further distinctly unique IARs, respectively. Still further, for flexion range of motion beyond the resting normal range, the IAR shifts generally cephalad, in a direction upward toward the superior bone of the joint (where superior bone is moving relative to the inferior bone). For the extension motion beyond normal resting range, the IAR shift is generally in a posterior direction.

These aspects and modes, and as further refined to certain of the more particular details such as just described, are considered broadly beneficial and novel to existing approaches to dynamic spinal stabilization. In addition, the particular embodiments herein presented in order to satisfy one or more of these aspects or modes are each also considered of particular benefit and typically in several regards. However, it is to be appreciated that other specific mechanical assemblies or implements may be provided other than those specifically herein shown and described, in order to accomplish the highly beneficial motion characteristics broadly presented by various aspects of the present disclosure. These broad aspects are thus to be considered of independent value and benefit, as are the more particular modes, embodiments, and features herein shown and described.

As mentioned elsewhere hereunder, further embodiments are contemplated though not herein specifically shown or described, and which are contemplated within the broad intended scope of the various aspects of the invention. Current data are for L5/S1, and motion patterns will be different for other spinal levels and thus accommodated in further embodiments properly responding from such information. Further embodiments are also to be modified and adapted as appropriate to also more closely approximate spinal motion under axial rotation and lateral bending, which particular dynamic is not specifically characterized in the motion according to the current data or resulting devices and methods for providing medical therapy herein described. In addition, improved generally applicable motion patterns (and in some circumstances more customized for particular parameters) may be further refined as more specimens are tested and in order to provide optimal prosthetic environment for assisting patients in restoring normal spinal motion.

Various of the embodiments herein shown and described are presented by illustrating a single mechanical assembly for providing dynamic stabilization. However, it is to be appreciated that in most applications for dynamic stabilization multiple such assemblies are implanted. For a single spinal joint level (e.g., two vertebra with a disc therebetween), two assemblies are often implanted, such as in posterior implantation with one on either posterolateral side of the spine so as to correspond with right and left pedicles of the respective vertebra. In addition, multiple spinal levels may be treated in a single patient using assemblies, or combinations thereof, of the present disclosure.

The following publications are herein incorporated in their entirety by reference thereto:
1. Adams M, Hutton W (1980). The effect of posture on the role of the apophysal joints in resisting intervertebral compressive force. J Bone Joint Surg 62-B(3): 358-362.
2. Adams M, Hutton W, Stott J (1980). The resistance to flexion of the lumbar intervertebral joint. Spine 5: 245-253.
3. Bertagnoli R, Kumar S (2002). Indications for full prosthetic disc arthroplasty: a correlation of clinical outcomes against a variety of indications. Eur Spine J 11 (S2): 131-136.
4. Buttermann G, Kahmann R, Lewis J, Bradford D (1991). An experimental method for measuring force on the spinal facet joint: description and application of the method. J Biomech Bioeng 113: 375-387.
5. Cholewicki J, Crisco J, Oxland T, Yamamoto I, Panjabi M (1996). Effects of posture and structure on three-dimensional coupled rotations in the lumbar spine. A biomechanical analysis. Spine 21: 2421-2428.
6. Cripton P, Bruehlmann S, Orr T, Oxland T, Nolte L (2000). In vitro preload application during spine flexibility testing: towards reduced apparatus-related artifacts. J Biomech 33: 1559-1568.
7. Dunlop R, Adams M, Hutton W (1984). Disc space narrowing and the lumbar facet joints. J Bone Joint Surg 66-B(5): 706-710.
8. Duval-Beaupere G, Robain G (1987). Visualization on full spine radiographs of the anatomical connections of the centers of the segmental body mass supported by each vertebra and measured in vivo. Int Orthp 11: 261-269.
9. Gertzbein S, Holtby R, Tile M, Kapasouri A, Chan K, Cruickshank B (1984). Determination of a locus of instantaneous centers of rotation of the lumbar disc by Moiré Fringes. A new technique. Spine 9(4): 409-413.
10. Gertzbein S D, Chan K H, Tile M, Seligman J, Kapasouri A (1985). Moire patterns: an accurate technique for determination of the locus of the centres of rotation. J Biomech 18(7): 501-509.
11. Granata K, Marras W (1995). An EMG-assisted model of trunk loading during free-dynamic lifting. J Biomech 28(11): 1309-1317.
12. Haher T, Bergman M, O'Brien M, Felmly W, Choueka J, Welin D, Chow G, Vassiliou A (1991). The effect of the three columns of the spine on the instantaneous axis of rotation in flexion extension. Spine 16(8S): S312-S318.
13. Kinzel G, Hall A, Hillberry B (1972). Measurement of the total motion between two body segments—I. Analytical development. J Biomech 5: 93-105.
14. Lazennec J, Ramaré S, Arafati N, Laudet C, Gorin M, Roger B, Hansen S, Sailllant G, Maurs L, Trablesi R (2000). Sagittal alignment in lumbosacral fusion: relations between radiological parameters and pain. Eur Spine J 9: 47-55.
15. Legaye J, Duval-Beaupere G, Hecquet J, Marty C (1998). Pelvic incidence: a fundamental parameter for three-dimensional regulation of spinal sagittal curves. Eur Spine J 7: 99-103.
16. Link HD (2002). History, design and biomechanics of the LINK SB Charite artificial disc. Eur Spine J 11 Suppl 2: S98-S105.
17. Lorenz M, Patwardhan A, Vanderby R (1983). Load-bearing characteristics of lumbar facets in normal surgically altered spinal segments. 1982 Volvo award in biomechanics. Spine 8(2): 122-130.
18. Mayer H, Wiechert K, Korge A, Qose I (2002). Minimally invasive total disc replacement: surgical technique and preliminary clinical results. Eur Spine J 11 (S2): 124-130.
19. Mc Glashen K, Miller J, Schultz A, Andersson G (1987). Load displacement behavior of the human lumbo-sacral joint. J Orthop Res 5(4): 488-496.
20. McGill S, Norman R (1987). Effects of an anatomically detailed erector spinae model on L4/L5 disc compression and shear. J Biomech 20(6): 591-600.
21. Nachemson A (1966). The load on lumbar disks in different positions of the body. Clin Orthop 45: 107-122.
22. Ogston N, King G, Gertzbein S, Tile M, Kapasouri A, Rubenstein J (1986). Centrode patterns in the lumbar spine. Baseline studies in normal subjects. Spine 11 (6): 591-595.
23. Oxland T, Crisco J, Panjabi M, Yamamoto I (1992). The effect of injury on rotational coupling at the lumbosacral joint. A biomechanical investigation. Spine 17(1): 74-80.
24. Panjabi M (1979). Centers and angles of rotation of body joints: a study of errors and optimization. J Biomech 12: 911-920.
25. Panjabi M, Oxland T, Yamamoto I, Crisco J (1994). Mechanical behavior of the human lumbar and lumbosacral spine shown by three-dimensional load-displacement curves. J Bone Joint Surg 76-A(3): 413-424.
26. Panjabi M, Yamamoto I, Oxland T, Crisco J (1989). How does posture affect coupling in the lumbar spine? Spine 14(9): 1002-1011. Order.
27. Pearcy M, Bogduk N (1988). Instantaneous axes of rotation of the lumbar intervertebral joints. Spine 13(9): 1033-1041.
28. Pearcy M, Portek I, Shepherd J (1984). Three-dimensional x-ray analysis of normal movement in the lumbar spine. Spine 9(3): 294-297.
29. Pearcy M J, Tibrewal S B (1984). Axial rotation and lateral bending in the normal lumbar spine measured by three-dimensional radiography. Spine 9(6): 582-587.
30. Posner I, White A, Edwrds W, Hayes W (1982). A biomechanical analysis of the clinical stability of the lumbar and lumbosacral spine. Spine 7(4): 374-389.
31. Sakamaki T, Katoh S, Sairyo K (2002). Normal and spondylolytic pediatric spine movements with reference to instantaneous axis of rotation. Spine 27(2): 141-145.
32. Schendel M, Wood K, Buttermann G, Lewis J, Ogilvie J (1993). Experimental measurement of ligament force, facet force, and segment motion in the human lumbar spine. J Biomech 26(4/5): 427-438.
33. Schultz A, Andersson G, Ortengren R, Bjork R, Nordin M (1982). Analysis and quantitative myoelectric measurements of loads on the lumbar spine when holding weights in standing postures. Spine 7(4): 390-397.
34. Seligman J, Gertzbein S, Tile M, Kappasouri A (1984). Computer Analysis of spinal segment motion in degenerative disc disease with and without axial loading. Spine 9(6): 566-573.
35. Shirazi-Adl A, Drouin G (1987). Load-bearing role of facets in a lumbar segment under sagittal plane loadings. J Biomech 20(6): 601-613.
36. Spoor C, Veldpaus F (1980). Rigid body motion calculated from spatial coordinates markers. J Biomech 13(4): 391-393.
37. Szpalski M, Gunzburg R, Mayer M (2002). Spine arthroplasty: a historical review. Eur Spine J 11 Suppl 2: S65-84.
38. Vaz G, Roussouly P, Berthonnaud E, Dimnet J (2002). Sagittal morphology and equilibrium of pelvis and spine. Eur Spine J 11 (1): 81-87.
39. White A, Panjabi M (1990). Clinical biomechanics of the spine, second edition. Lippincott Company, Philadelphia.
40. Woltring H, Huiskes R, De Lange A (1985). Finite centroid and helical axis estimation from noisy landmark measurement in the study of human joint kinematics. J Biomech 18(5): 379-389.
41. Yamamoto I, Panjabi M M, Crisco T, Oxland T (1989). Three-dimensional movements of the whole lumbar spine and lumbosacral joint. Spine 14(11): 1256-1260.
42. Yamamoto I, Panjabi M M, Oxland T R, Crisco J J (1990). The role of the iliolumbar ligament in the lumbosacral junction. Spine 15(11): 1138-1141.
43. Yoshioka T, Tsuji H, Hirano N, Sainoh S (1990). Motion characteristics of the normal lumbar spine in young adults: instantaneous axis of rotation and vertebral center motion analyses. J Spinal Disord 3(2): 103-113.

The following issued US Patents are also herein incorporated in their entirety by reference thereto issued U.S. Pat. Nos. 4,932,975; 4,966,599; 5,129,899; 5,242,443; 5,474,551; 5,480,440; 5,486,176; 5,499,983; 5,545,228; 5,558,674; 5,584,887; 5,620,443; 5,643,260; 5,643,265; 5,827,328; 5,885,299; 5,891,060; 5,928,243; 5,935,133; 5,954,674; 5,964,769; 5,989,250; 5,989,251; 6,030,389; 6,053,921; 6,066,140; 6,080,193; 6,083,224; 6,132,430; 6,206,882; 6,224,631; 6,254,603; 6,287,343; 6,302,882; 6,368,321; 6,391,030; 6,391,058; 6,413,257; 6,416,515; 6,454,769; 6,471,704; 6,491,702; 6,533,786; 6,562,040; 6,576,016; 6,595,992; 6,602,254; 6,613,050; 6,641,614; 6,679,883; 6,682,533; 6,692,503; 6,701,174; 6,711,432; 6,716,214; 6,725,080; 6,770,075; 6,783,527; 6,887,241; 6,926,718; 6,932,820; 6,936,050; 6,936,051; and 6,947,786.

The following published US Patent Applications are also herein incorporated in their entirety by reference thereto (US Published Patent Application Number): 2001/0010000; 2002/0052603; 2002/0072753; 2002/0193795; 2003/0083658; 2003/0130661; 2004/0186475; 2004/0220672; 2005/0033298; 2005/0113924; 2005/0113927; 2005/0143737; 2005/0143823; 2005/0171543; 2005/0177156; 2005/0177157; 2005/0177164; 2005/0177166; 2005/0182400; 2005/0182401; and 2005/0182409.

The following published PCT International Patent Applications are also herein incorporated in their entirety by reference thereto (PCT Patent Application Publication Number): WO 99/65414; WO 00/19923; WO 00/57801; WO 01/52758; WO 02/11650; WO 03/037169; WO 05/013852; WO 05/053572; WO 05/062902; 05/065374; WO 05/065375; WO O 05/084567; and WO 05/087121.

The issued patents, published patent applications, articles, and other published references that are herein incorporated by reference thereto are to be considered in the context of this overall disclosure, and are incorporated to the extent consistent with this disclosure in a manner providing additional context to the present embodiments, and are otherwise incorporated and considered as general information and background with respect to the context of the present invention as described in various details by reference to the particular embodiments and figures herein disclosed and shown. In particular, it is to be appreciated that these incorporated disclosures are to be considered against the context of various information disclosed here that is believed to be newly informative with respect to normal spinal motion, and with respect to prosthetic device assemblies and methods adapted to approximate that motion as improved medical tools versus the previously disclosed alleged solutions.

Although the description above contains many details, these should not be construed as limiting the scope of the invention but as merely providing illustrations of some of the presently preferred embodiments of this invention. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

TABLE I
Direction (inclination and declination) and coordinates of the IAR intersection with
the midsagittal plane in flexion/extension (xᵢ, yᵢ, zᵢ). Averages ± standard error.
Flexion/Extension:

| motion sector | inclination (°) | declination (°) | xᵢ (mm) | yᵢ (mm) | zᵢ (mm) |
|---|---|---|---|---|---|
| 3-6° extension | 2.2 ± 0.9 | 91.6 ± 1.1 | −12.3 ± 1.70 | 0 | 4.6 ± 3.1 |
| 0-3° extension | 1.7 ± 0.8 | 90.6 ± 1.0 | −8.8 ± 1.3 | 0 | 6.7 ± 1.3 |
| 0-3° flexion | 0.3 ± 0.8 | 92.0 ± 0.9 | −6.8 ± 1.2 | 0 | 5.7 ± 1.7 |
| 3-6° flexion | 1.0 ± 0.7 | 91.6 ± 0.8 | −7.3 ± 0.9* | 0 | 13.5 ± 2.2* |

*significant variation: the IAR moved up in flexion and backward in extension.

TABLE II
Facet force in flexion/extension. Averages ± standard error.
Flexion/Etension:

| Posture | average facet force (N) |
|---|---|
| 6° extension | 54.1 ± 14.6 |
| 3° extension | 51.6 ± 12.6 |
| Neutral | 50.4 ± 9.8 |
| 3° flexion | 47.6 ± 10.6 |
| 6° flexion | 43.9 ± 9.0 |

TABLE III
Direction (inclination and declination) and coordinates of the IAR position in
lateral bending (xₚ, yₚ, zₚ). Averages ± standard error.
Lateral Bending:

| motion sector | inclination (°) | declination (°) | xₚ (mm) | yₚ (mm) | zₚ (mm) |
|---|---|---|---|---|---|
| 3-6° left | 21.0 ± 6.4 | 1.6 ± 0.4 | −11.2 ± 1.3 | −6.7 ± 1.2 | 19.2 ± 1.6 |
| 0-3° left | 17.7 ± 5.2 | 1.4 ± 0.3 | −6.7 ± 1.5 | −1.1 ± 1.0 | 13.9 ± 2.3 |
| 0-3° right | 27.0 ± 2.8 | 2.1 ± 0.4 | −9.6 ± 2.2 | −2.0 ± 2.1 | 15.8 ± 2.6 |
| 3-6° right | 33.3 ± 2.5* | 2.1 ± 0.4 | −13.9 ± 2.1* | −9.7 ± 1.6* | 20.8 ± 2.8* |

*significant variation: the IAR moves up backward and in the bending direction in lateral bending; inclination increases beyond 3° lateral bending.

TABLE IV
Facet force in lateral bending. Averages ± standard error.
Lateral Bending:

| Posture | average facet force (N) |
|---|---|
| −6° | 20.7 ± 6.7 |
| −3° | 22.0 ± 4.5 |
| Neutral | 36.9 ± 5.8 |
| 3° | 68.2 ± 15.9 |
| 6° | 58.4 ± 16.1* |

*significant increase in the facet force ipsilaterally to the bending between neutral and 3° bending.

What is claimed is:

1. A method of stabilizing adjacent vertebrae, comprising:
installing a first anchor in a first vertebra;
installing a second anchor in a second vertebra;
said second vertebra being adjacent to said first vertebra; and
coupling an articulating linkage to said first and second anchors;
wherein said articulating linkage constrains one or more components of motion between the first and second vertebrae while allowing the first vertebra to move substantially along the path of an instantaneous axis of rotation (IAR) associated with the first vertebra.

2. A method as recited in claim 1, wherein the linkage is configure to constrain non-physiologic motion between the first and second vertebrae.

3. A method as recited in claim 1, wherein the IAR of the first vertebra comprises an axis that the first vertebra rotates about and travels along as it moves from one position to another.

4. A method as recited in claim 1, wherein coupling an articulating linkage to said first and second anchors comprises:
attaching a first member to the first anchor;
attaching a second member to the second anchor; and
establishing one or more hinges about one or more respective pivot points;
said one or more hinges linking the first member to the second member;
wherein the one or more pivot points correlate to the IAR of the first vertebra.

5. A method as recited in claim 4, wherein the first member is coupled to the second member via a first articulating link having a first pivot point on the first member and a second pivot point on the second member, and a second articulating link having a third pivot pint on the first member and a fourth pivot point on the second member.

6. A method as recited in claim 4:
wherein the first member and first anchor are rigidly fixed to each other such that they move in unison along with the first vertebra; and
wherein the second member and second anchor are rigidly fixed to each other such that they move in unison along with the second vertebra.

7. A method as recited in claim 1, wherein installing a first anchor comprises installing a pedicle screw in a pedicle of the first vertebra.

8. A method as recited in claim 1, wherein the linkage is installed in a posterior region of the vertebrae.

9. A method as recited in claim 1:
wherein the first vertebra comprises the L5 vertebra; and
wherein the second vertebra comprises the S1 vertebra.

10. A method as recited in claim 9:
wherein the articulating linkage allows the L5 vertebra to rotate and translate with respect to the S1 vertebra.

11. A method as recited in claim 10, wherein the rotation and translation of the L5 vertebra follows that path of the IAR of the L5 vertebra.

12. A method as recited in claim 9:
wherein the L5 IAR intersection with the midsagittal plane moves cephalid relative to the S1 endplate during flexion, and posterior during extension.

13. A method as recited in claim 9:
wherein the articulating linkage is configured to allow the L5 vertebra to rotate substantially forward during flexion, and substantially backward during extension.

14. An apparatus for stabilizing adjacent vertebrae, comprising:
a first anchor configured to be installed in a first vertebra;
a second anchor configured to be installed in a second vertebra;
said second vertebra being adjacent to said first vertebra; and
an articulating linkage coupling said first and second anchors;
wherein said articulating linkage is configured to constrain one or more components of motion between the first and second vertebrae while allowing the first vertebra to move substantially along the path of an IAR associated with the first vertebra.

15. An apparatus as recited in claim 14, wherein the linkage is configured to constrain non-physiologic motion between the first and second vertebrae.

16. An apparatus as recited in claim 14; wherein the IAR of the first vertebra comprises an axis that the first vertebra rotates about and travels along as it moves from one position to another.

17. An apparatus as recited in claim 14, wherein the articulating linkage comprises:
a first member configured to be attached to the first anchor;
a second member configured to be attached to the second anchor; and
one or more hinges centered about one or more respective pivot points;
said one or more hinges linking the first member to the second member;
wherein the one or more pivot points correlate to the IAR of the first vertebra.

18. An apparatus as recited in claim 17, wherein the first member is coupled to the second member via a first articulating link having a first pivot point on the first member and a second pivot point on the second member, and a second articulating link having a third pivot point on the first member and a fourth pivot point on the second member.

19. An apparatus as recited in claim 17:
wherein the first member and first anchor are rigidly fixed to each other such that they are configured to move in unison along with the first vertebra; and
wherein the second member and second anchor are rigidly fixed to each other such that they are configured to move in unison along with the second vertebra.

20. An apparatus as recited in claim 14:
wherein the first anchor comprises a first pedicle screw configured to be installed in a pedicle of the first vertebra; and
wherein the second anchor comprises a second pedicle screw configured to be installed in a pedicle of the second vertebra.

21. An apparatus as recited in claim 14, wherein the articulating linkage is configured to be installed in a posterior region of the vertebrae.

22. An apparatus as recited in claim 14:
wherein the first vertebra comprises the L5 vertebra; and
wherein the second vertebra comprises the S1 vertebra.

23. An apparatus as recited in claim 22:
wherein the articulating linkage is configured to allow the L5 vertebra to rotate and translate with respect to the S1 vertebra.

24. An apparatus as recited in claim 23, wherein the rotation and translation of the L5 vertebra follows that path of the IAR of the L5 vertebra.

25. An apparatus as recited in claim 22:
wherein the L5 IAR intersection with the midsagittal plane moves cephalid relative to the S1 endplate during flexion, and posterior during extension.

26. An apparatus as recited in claim 22:
wherein the articulating linkage is configured to allow the L5 vertebra to rotate substantially forward during flexion, and substantially backward during extension.

27. An apparatus for dynamically stabilizing adjacent vertebrae, comprising:
a superior anchor configured to be installed in a superior vertebra;
an inferior anchor configured to be installed in an inferior vertebra;
said inferior vertebra being adjacent to said superior vertebra; and
means for rotatably linking the superior anchor with the inferior anchor such that one or more components of motion between the superior and inferior vertebrae are constrained while allowing the superior vertebra to move along the path of an IAR associated with the superior vertebra.

28. An apparatus as recited in claim 27, wherein said means is configured to constrain non-physiologic motion between the first and second vertebrae.

29. An apparatus as recited in claim 27, wherein said means articulates about one or more pivot points that correlate to the instantaneous axis of rotation (IAR) of the first vertebra.

30. An apparatus as recited in claim 27:
wherein the superior anchor comprises a superior pedicle screw configured to be installed in a pedicle of the superior vertebra; and
wherein the inferior anchor comprises a inferior pedicle screw configured to be installed in a pedicle of the inferior vertebra.

31. An apparatus as recited in claim 30:
wherein the superior vertebra comprises the L5 vertebra; and
wherein the inferior vertebra comprises the S1 vertebra.

32. An apparatus as recited in claim 31:
wherein the linking means is configured to allow the L5 vertebra to rotate and translate with respect to the S1 vertebra.

33. An apparatus as recited in claim 32, wherein the rotation and translation of the L5 vertebra follows that path of the IAR of the L5 vertebra.

34. An apparatus as recited in claim 32:
wherein the L5 IAR intersection with the midsagittal plane moves cephalid relative to the S1 endplate during flexion, and posterior during extension.

35. An apparatus as recited in claim 32:
wherein the linking means is configured to allow the L5 vertebra to rotate substantially forward during flexion, and substantially backward during extension.

36. An apparatus for stabilizing first and second adjacent vertebrae, comprising:
a dynamic stabilization assembly configured to be implanted in relation to the first and second vertebrae;
wherein the first and adjacent vertebra comprise a vertebral joint having at least one instantaneous axis of rotation (IAR) associated with the first and second vertebrae; and
wherein the dynamic stabilization assembly is configured to allow at least a portion of the vertebral joint to rotate substantially about a first IAR corresponding to a first range of motion associated with the vertebral joint.

37. An apparatus as recited in claim 36:
wherein the dynamic stabilization assembly is further configured to allow at least a portion of the vertebral joint to rotate substantially about a second IAR corresponding to a second range of motion associated with the vertebral joint.

38. An apparatus as recited in claim 37, wherein the first IAR and second IAR have different locations with respect to a disc plane associated with the vertebral joint.

39. An apparatus as recited in claim 38, wherein the position of the first IAR with respect to the second IAR shifts substantially laterally across the disc plane during the first range of motion.

40. An apparatus as recited in claim 39, wherein the position of the first IAR with respect to the second IAR shifts substantially vertically along a spinal axis of the first and second vertebrae during the second range of motion.

41. An apparatus as recited in claim 36:
wherein the dynamic stabilization assembly comprises a plurality of members coupled to the first and second vertebrae; and
wherein the plurality of members are configured to constrain motion of the vertebral joint while allowing at least a portion of the vertebral joint to move in accordance with the IAR.

42. An apparatus as recited in claim 41, wherein the plurality of members comprise a four-bar linkage.

43. An apparatus as recited in claim 42, wherein the linkage comprises a plurality of pivot points associated with the IAR.

44. A method for stabilizing first and second adjacent vertebrae, comprising:
implanting a dynamic stabilization assembly in relation to the first and second vertebrae;
wherein the first and adjacent vertebra comprise a vertebral joint having at least one instantaneous axis of rotation (IAR) associated with the first and second vertebrae; and
restraining motion of the vertebral joint while allowing at least a portion of the vertebral joint to rotate substantially about a first IAR corresponding to a first range of motion associated with the vertebral joint.

45. A method as recited in claim 44:
wherein the dynamic stabilization assembly is configured to allow at least a portion of the vertebral joint to rotate substantially about a second IAR corresponding to a second range of motion associated with the vertebral joint.

46. A method as recited in claim 45, wherein the first IAR and second IAR have different locations with respect to a disc plane associated with the vertebral joint.

47. A method as recited in claim 46, wherein the position of the first IAR with respect to the second IAR shifts substantially laterally across the disc plane during the first range of motion.

48. A method as recited in claim 46, wherein the position of the first IAR with respect to the second IAR shifts substantially vertically along a spinal axis of the first and second vertebrae during the second range of motion.
